(19) United States
(12) Patent Application Publication
(10) Pub. No.: US 2002/0068051 A1
(43) Pub. Date: Jun. 6, 2002

(54) Generation and use of signal-plexes to develop specific cell types, tissues and /or organs

(76) Inventors: Jianwu Dai, Boston, MA (US); Eugene Bell, Boston, MA (US)

Correspondence Address: Ellen M. Leonnig, TEI Biosciences, Inc., 7 Elkins Street, Boston, MA, 02127, US

(21) Appl. No.: 09/901,765

(22) Filed: Jul. 9, 2001

Related U.S. Application Data
(60) Provisional application No. 60/256,614, filed on Dec. 18, 2000.
(60) Provisional application No. 60/256,593, filed on May 29, 2001.
(60) Provisional application No. 60/251,125, filed on Dec. 4, 2000.

(51) Int. Cl. A61K 48/00; A61K 35/00

(52) U.S. Cl. 424/93.21; 424/115

(57) ABSTRACT

This invention includes methods and compositions for generating signaling complexes (“Signal-plexes”) and the use of Signal-plexes for inducing cell divisions, differentiation and transdifferentiation of cells into specific cell, tissue or organ types which resemble the cells, tissues or organs from which the Signal-plexes were derived.

Signal-plexes may be used to induce the development of any specific cells, tissues or organs of the body. Signal-plexes may be derived from any tissue of the body. Signal-plexes may be combined with stem cells or cells having stem cell properties in scaffolds for differentiation into tissue or organs in vitro and or in vivo.

The applications of this invention comprise tissue repair, and tissue or organ regeneration. Signal-plexes may be used as pharmaceutical agents; they may be used in humans as well as non-humans.

### CROSS-REFERENCE TO RELATED APPLICATIONS

This application is related to co-pending U.S. Application No(s). 60/256,614, filed Dec. 18, 2000, 60/256,593, filed Dec. 18, 2000 and 60/251,125 (from which it claims priority), filed Dec. 4, 2000, and a U.S. Application filed concurrently, entitled “Use of Stem Cells Derived from Fetal Skin,” the entire contents of which are incorporated herein by reference.

### BACKGROUND OF THE INVENTION

1. Field of the Invention

In searching for new ways to restore failed or failing body parts, scientists studying the areas of regenerative medicine and tissue engineering turn more and more toward stem cells as a resource. While there has been progress in understanding how stem cells may be best used, and in probing the questions of the relative value of choosing embryonic, fetal or adult stem cells, the very basic requirement that stem cells need precise instructions to become a particular part of the body has remained unsatisfied.

Signaling in tissue development is believed to be a holistic process involving multiple signals. Complexes of tissue-specific signals are present and active in the course of tissue and organ development in the embryo and fetus, and that the nature of these signaling complexes in the tissues change as development progresses.

Signaling complexes are tissue-specific compositions comprising a number of factors necessary to promote cell division, direct patterning, morphogenesis and differentiation of specific cells, tissues and organs. During early animal development, different tissues and organs contain specific pools of signaling molecules, loosely called growth factors. It is believed that 1) the particular factors present in signaling complexes; 2) the relative proportions of these factors to one another, e.g., the proportions of the factors found in vivo, are responsible for cell divisions, morphogenesis, differentiation, histiogenesis and organogenesis; and 3) the time spans over which they function. Despite the significance of signaling complexes, other factors, namely transcription factors activated by signaling complexes, also contribute to the developmental process.

2. Description of the Related Art

Cytokines and other signaling molecules have been shown to play a key part in initiating and accelerating tissue development, but the principal approach has been based mainly on the use of high doses of usually a single human recombinant product, at high cost. Another approach depends on the insertion of a cytokine gene, to upregulate output of a particular cytokine capable of improving tissue repair by improving vascular competence for example. Although there have been some successes with the foregoing approaches, neither is fully physiologic.

Embryonic stem cells, fetal stem cells and adult stem cells share great promise in the field of tissue engineering. A major difficulty standing in the way of their most effective use is the need for signaling to direct the replication, morphogenesis and differentiation of embryonic, fetal and adult stem cells and of cells of any age capable of responding to signals able to induce transdifferentiation or signals able to accelerate tissue building and repair. Individual growth factors have been tested on mouse stem cells and human stem cells to identify the instructive molecules required to guide stem cell differentiation (Rohwedel et al, 1994, Dev. Biol, 164, 87-101; Schuldiner et al, 2000, PNAS, 97, 11307-11312). However, determining the full panel of factors in use at each stage of tissue ontogenesis by trial-and-error is an extremely long term process, particularly since a myriad of specific factors have not yet been identified.

### BRIEF DESCRIPTION OF THE INVENTION

Signaling complexes may be used for inducing stem and other cells to make new body parts in vitro or to regenerate failing or malfunctioning body parts in vivo. Tissues or organs produced in vitro may then be grafted to a recipient.

These signaling complexes are thought of as a family (“Signal-plexes”), each member of which exhibits specificity with respect to the kind of cells, tissues or organs it is capable of inducing in stem cell populations.

Specifically, Signal-plexes may be used: 1) in combination with cells and scaffolds ex vivo to create an implantable tissue or organ precursor; 2) in combination with a scaffold alone with the expectation that after implantation, the scaffold will not only be vascularized, but also populated by relevant host cells; and or 3) alone as a pharmaceutical agent deliverable by injection alone or with a carrier (e.g., matrix of hydrated collagen fibers). In a suitable carrier, such as a salve or ointment, the pharmaceutical agent can be applied to an exterior or interior surface of any body part.

### DETAILED DESCRIPTION OF THE INVENTION

Signal-plexes provided by the methods herein, have broad applications for directing stem cells and other cells to form specific cells, tissues and organs.

Signal-plexes may be used to predictably direct the differentiation of embryonic or fetal stem cells, or of cells which appear to be committed, and are destined to become specialized because of their position anatomically, or of cells because of their association with other cells which are or appear to have become specialized, but in fact may have stem cell properties, or of cells which have become specialized to a degree but nonetheless are still capable of being transdifferentiated (cells whose fates have been determined but are reversible), or of cells in the adult bone marrow known to be a mixed population of stem cells which have a broad but still limited repertoire of phenotypes or of other sources of stem cells in the embryo, fetus or adult. Thus, a Signal-plex can be designed to induce stem cells or cell types of any age to adopt the phenotype of the tissue from which the Signal-plex comes.

Signal-plexes may be used to promote the growth of any type of animal tissue or organ, from nervous tissue, skin, vascular tissue, cardiac tissue, pericardial tissue, muscle tissue, ocular tissue, periodontal tissue, connective tissue (e.g. bone, cartilage, tendon or ligament tissue), organ tissue (e.g., kidney or liver tissue), glandular tissue (e.g., pancreatic, mammary or adrenal tissue), urological tissue (e.g., bladder or ureter tissue), and digestive tissue (e.g. intestinal tissue). Similarly, Signal-plexes may be derived from any tissue or organ of an animal's body (e.g., brain, nerve, skin, heart, vascular, liver, kidney, pancreas, lung, bone, cartilage, tendon, cardiac, pericardial, muscle, ocular, periodontal, connective, pancreatic, mammary, adrenal, urological, digestive or ligament tissue).

The use of Signal-plexes is not limited to repair or rebuilding of a specific tissue or organ. For example, specific fractions of heart or lung extracts may also be used for repairing or regenerating other types of tissues, as well as the heart or lung specifically, since both promote angiogenesis. They may be used to promote regeneration of heart tissue before or after a heart attack or regeneration of lung tissue

Combinations of fractions from one or more complexes may be used to induce particular features of wound healing and tissue or organ rebuilding. Thus, extracts or fractions generated may have broad use for rebuilding or repairing any tissue or organ by providing additional signals which induce or optimize specific repairatory effects.

Tissue-specific animal extracts, preferably extracts from developing fetal tissues or organs as well as fractions thereof, may be used. Signaling complexes and or fractions thereof include those extracted from tissue at specific developmental stages. Extracts are compositions or mixtures derived from freeze-drying, breaking, lysing, or homogenizing the tissue cells and subjecting the lysate to any number of fractionation techniques, as described herein. In a preferred embodiment, extracts are made from newborn or fetal animal tissue. In another embodiment, Signal-plexes are extracted from tissue extracellular matrix particulates derived from specific tissues as described in U.S. patent application Ser. No. 60/251,125 referred to herein. In an alternative embodiment, the source of the extracts are tissue-specific microparticulates prepared by the method described in U.S. Pat. No. 5,800,537, the entire contents of which are herein incorporated by reference. Preferably, an extract of the invention does not contain cellular membranes or nucleic acids (e.g., DNA or RNA).

Animal tissue extracts and fractions that retain the specificity of the signaling complexes present during particular stages of tissue and organ development may be identified as described herein. The directive or inductive capacity of extracts and fractions containing selected signaling complexes are tested on stem cell populations to assess their specificity. The search has been that of discovering the specific fractions of a tissue extract capable of optimally inducing cells to adopt phenotypes which are the same as the phenotype of the source tissue of the extract.

Undifferentiated embryonic stem cells or stem cells aggregated into embryoid bodies (EB) and combined with Signal-plexes before they differentiate have been used as test systems for determining the specificity of a signaling complex to direct fractions of a tissue extract capable of optimally inducing cells to adopt phenotypes which are the same as the phenotype of the source tissue of the extract.

Undifferentiated embryonic stem cells or stem cells aggregated into embryoid bodies (EB) and combined with Signal-plexes before they differentiate have been used as test systems for determining the specificity of a signaling complex to direct tissue-specific differentiation in vitro or in vivo. If EBs are to be used in vivo, the stem cells and the signaling complex are combined in a collagen scaffold or any other type of scaffold before implantation to produce a tissue or organ primordium which can then be grafted to a host.

In a preferred embodiment, Signal-plexes may be used to identify an undefined subset of cells which reside in the dermis of fetal skin. Using extracts of Signal-plexes from fetal porcine cartilage, bone, muscle, endocrine or exocrine pancreas tissue, these fibroblastic cells can be differentiated into functional cartilage, osteoblasts, muscle cells or insulin-secreting cells. The process involves isolating the fibroblasts from 8-week to 24-week human fetal skin. After two passages, the cells are then cultured in a three dimensional collagen scaffold with low serum medium or defined medium with the addition of either cartilage, bone, muscle, or pancreas extract at a total protein concentration of 1.0 pg/ml to 20 mg/ml. The culture medium is changed every 3-4 days.

After a week in culture, the cells undergo dramatic morphological changes. Both RT-PCR and immunostaining are used to characterize muscle cells and insulin-secreting cells at both mRNA and protein levels. Specifically, muscle actin is one of the markers for muscle cells, and insulin is one of the markers for insulin-secreting cells.

### EXAMPLE 1

Generation of Signal-plexes

Preferred, non-limiting examples of procedures for generating Signal-plexes are as follows.

Signal-plexes may be extracted from tissue by buffer extraction. Tissue is collected from fetal animals or newborn animals, washed with buffer, and cut into small pieces. The buffer may be, for example, Tris buffer (at approximately a pH of 4.0-11.0), HEPES buffer (at approximately a pH of 7.0-8.0 and preferably, e.g., 7.4), or PBS (at approximately a pH of 7.0-8.0 and preferably, e.g., 7.4). The buffer preferably includes EDTA (at, e.g., 0-10 mM, 0.5-5 mM, or preferably, e.g., 2 mM), and may also include protease inhibitors (e.g., 1 mM PMSF and or 1 μM E-64). Preferably, the buffer is cold (e.g., at approximately 4° C.). The previously frozen and thawed microparticulates or cut pieces of tissue are homogenized in buffer (preferably, e.g., the same buffer that was used for washing), and extracts are obtained by collecting the supernatant after centrifugation at, for example, 17,000 g for about 20 minutes, to remove particulate matter, including mitochondria.

Signal-plexes may be extracted from tissue by enzyme extraction. Enzymes are used to degrade the extracellular matrix (e.g., extracellular matrix proteins, such as collagen) to release any signaling molecules that bind to the matrix. Homogenized tissue is incubated with an enzyme and then centrifuged at, e.g., 17,000 g for about 20 minutes to remove particulate matter such as mitochondria. The signaling complexes are recovered from the supernatant. A preferred example of enzyme extraction is as follows: homogenized tissue is incubated with 180 U/ml hyaluronidase at room temperature for approximately 1.5 hours and then with 160 U/ml collagenase 4:3 for approximately 1.5 hours at room temperature. Those skilled in the art will recognize that any enzyme that can dissociate or degrade the extracellular matrix may be used.

Signal-plexes may also be extracted from extracellular matrix particulates by acid extraction. This method is used to extract Signal-plexes which are soluble at low pH. A preferred, non-limiting example is as follows: 0.2 ml of, eg., 1 N HCl is added to each ml of the tissue homogenate; the mixture is mixed for approximately 30 minutes at room temperature; and, the extract is neutralized with 10 N NaOH by titration.

Other fractionation techniques may be employed in generating Signal-plexes, such as chromatographic or separation techniques including ion exchange (e.g., anion or cation exchange) chromatography, gel filtration chromatography, affinity chromatography, high-performance liquid chromatography (HPLC), capillary electrochromatography (CEC), gradient (e.g., glycerol or sucrose gradient) centrifugation, dialysis, two-dimensional gel electrophoresis, immunoprecipitation, and ammonium sulfate precipitation. The final product may be stored or used, for example, in the form of a solution or a lyophilized powder.

### EXAMPLE 2

Identification of Signal-plexes

Methods for identifying or screening Signal-plexes from animal tissue extracts or fractions thereof are disclosed herein. These methods are helpful for identifying Signal-plexes which can direct differentiation of stem cells and or transdifferentiation of cells which are not stem cells into specific cell types, tissues or organs.

In one embodiment, pluripotent murine embryonic stem (ES) cells are used to assay tissue extracts and or fractions thereof for their ability to direct the differentiation of ES cells into specific cell types. Another approach uses ES cells which are first assembled into embryoid bodies (EBs) using methods well-known in the art; however, a medium containing the Signal-plexes is added to the culture when the EB is formed. Like the ES cells, the EB cells may be dissociated and cultured in the presence of various tissue extracts and or fractions thereof. In a preferred embodiment, the EB cells induced by the addition of Signal-Plexes are cultured in three dimensions, for example in a collagen scaffold with a defined medium or low serum medium. ES or EB cells may also be cultured as a monolayer culture or in suspension. The period of time the ES or EB cells are cultured may range from about 1 week to about 6 months.

At the end of the culture period, the cells are assayed for the cell or tissue types from which the Signal-plexes are derived. The cells may be assayed for the presence of one or more cell or tissue-specific marker by, e.g., immunofluorescence or ELISA. In one embodiment, cells with their three dimensional scaffolds may be processed for histological analysis. In another embodiment, the cells may be assayed for expression of one or more tissue-specific mRNAs using Northern blotting or RT-PCR, methods which are well-known in the art. Tissue extracts or fractions thereof which can induce differentiation of ES or EB cells into specific cell types are thus identified as signaling complexes which can be used in the compositions and methods of the invention.

### EXAMPLE 3

Use of Signal-plexes for Differentiation

A preferred embodiment is the use of signaling complexes for cell differentiation. Signal-plexes of the invention can be used to redirect or differentiate fetal or adult cells to adopt new phenotypes and develop into tissues and or organs.

Cells which have been genetically altered and cells which are taken from a donor may be used. They may be cultivated or not cultivated in vitro and differentiated or transdifferentiated for return to the donor to provide a replacement part or cell type which the donor lacks. Alternatively, the differentiated cells are used to create an organ or tissue primordium for implantation to the donor for tissue or organ repair or replacement.

Cells may be cultured using any number of culture methods (e.g., monolayer or three-dimensional culture). Extracts and or fractions thereof can be applied in vitro (e.g., added to the culture), and may also by applied in vivo (e.g., added during implantation of a primordium).

For example, human fetal skin fibroblasts can be transdifferentiated into liver cells using liver extracts from fetal pigs. Fibroblasts are first isolated from 8-week to 24-week human fetal skin. After two passages, the cells are cultured in a three-dimensional collagen scaffold with either low serum medium or defined medium with the addition of either muscle extract or pancreas extract at a total protein concentration of 1.0 pg/ml to 20 mg/ml. The culture medium is changed every 3-4 days. After a week in culture, morphological changes can be observed in the cells; RT-PCR and or immunostaining can be used to characterize muscle cells and insulin-secreting cells at both mRNA and protein levels. Specifically, albumin is a marker for liver cells.

### EXAMPLE 4

Use of Signal-plexes for Tissue or Organ Regeneration

Generally, the methods herein for repairing tissues and or regenerating tissues and organs feature two steps: 1) combining stem cells with biocompatible matrix material in a three dimensional scaffold (e.g., collagen) and Signal-plexes to form tissue or organ primordia in vitro; if host cells are available in the vicinity of the graft, seeding the primordium with cells may be unnecessary for some types of implants; and 2) implantation of tissue or organ primordia for in vivo tissue development and regeneration. These methods may be used to repair and or regenerate any tissue or organ of the body (e.g., skin, liver, kidney, pancreas, blood vessel, bone, cartilage, ligament, and tendon).

A variety of collagen-based scaffolds exist that are suitable for regeneration of many types of tissues and target organs (see, e.g., U.S. Pat. Nos. 5,800,537; 5,709,934; 5,893,888; and 6,051,750, the entire contents of which are herein incorporated by reference). Such scaffolds provide a biocompatible substrate to which intermediate binding molecules such as heparin or heparin sulfate as well as Signal-plexes can bind cells.

The scaffolds used may be, e.g., cross-linked and freeze-dried collagen or collagen fiber, collagen gel, a collagen-gel mixture, or any of these with the addition of different forms of collagen (e.g., dense fibrillar collagen), or the addition of other types of proteins or polymers (e.g., gelatin). The cross-linking procedure for scaffolds may be carried out by using a variety of chemical or physical cross linkers, such as, genipin or UV irradiation respectively. Thus, different types of biocompatible scaffolds having mechanical and other physical and chemical properties suitable for different types of tissue regeneration may be chosen.

The various collagen scaffolds provide a three dimensional structure to which cells can attach and grow and resemble the native microenvironments which favor cell differentiation and tissue development. The methods of adding the cells to the scaffolds may vary. Cells may be added to freeze-dried scaffolds by hydrating the scaffolds with a cell suspension (e.g., at a concentration of about 100 to 1 million or more cells/ml of medium). Incorporation of cells into other types of scaffolds may be carried out by adding cells to the collagen solution, preferably at 4° C. The methods of adding the Signal-plexes to the scaffolds may vary. Signal-plexes may be added when the freeze-dried scaffolds are manufactured or when tissue extracts or fractions thereof are added to the culture directly. Signal-plexes may be added to a collagen solution or culture medium.

Low serum medium or defined medium may be used preferably for in vitro stem cell differentiation and or cell transdifferentiation. When using small scaffolds (<100 mm³ in size), the medium is changed manually, and the Signal-plexes are added every 3-4 days. When using larger scaffolds, the culture may be maintained in a bioreactor system. The system is designed to use a minipump for medium change. The pump is operated in the incubator. Scaffolds are kept in a special container with two tubes connected to the pump. Out of the scaffold container, fresh medium is mixed with the medium pumped out. The medium pumped back to the container will container about 5% fresh medium. This ratio varies from about 1% fresh medium to about 50% fresh medium. When Signal plexes are added, 100% fresh medium containing these Signal-plexes are added to the scaffold. The pump rate is adjusted to approximately 0.1 ml/min or slower. The medium delivery system can be tailored to the type of tissue being manufactured. All culture is performed under sterile conditions.

### EXAMPLE 5

Grafts with Signal-plexes

After grafting cells and Signal-plexes to an animal or human host, vascularization is critical to the success of the grafted tissue. In one embodiment, Signal-plexes that promote vascularization in vitro may be used. In another embodiment, a primordium is implanted into an animal host or directly into a human to allow tissue development and organ regeneration to occur under native conditions. If using an animal host in which human cells are to be tested, it is necessary to use an immunodeficient animal or animals as are known in the art of implantation. In human subjects, if the transplanted tissue is originally derived from the subject's own cells, immunosuppressive drugs are not needed. If the transplanted tissue is originally derived from cells from a different subject, immunosuppressive drugs or agents may or may not be necessary.

### EXAMPLE 6

Use of Signal-plexes for Wound Healing

In another embodiment of the invention, Signal-plexes are used for wound healing. For example, fetal skin tissue extracted with Tris-buffer yields an extract that can be used to treat topical wounds (e.g., skin wounds). In practicing this invention, the inventors have found that one application of Signal-plexes results in significant reduction of wound contraction in a rat model, compared with control grafts.

Signal-plexes may be delivered to the wound in a carrier matrix, for example, a cross-linked collagen scaffold, a collagen foam, an injectable collagen fiber as referred to herein, and in an EBM scaffold (as described in a co-pending U.S. patent application filed on May 31, 2001, the entire contents of which are herein incorporated by reference), or in a salve, ointment or emollient. Treatment may consist of one or more applications of these carriers to heal a single wound. In one embodiment, the treatment includes application of one or more grafts of the carrier matrix containing the extract to treat a single wound. In another embodiment, one graft is used, and multiple doses of the extract can be given by successive applications or injections to the graft.

Before the treatment, the carriers are hydrated with a solution of the tissue extract containing the Signal-plexes (the total protein concentration ranges from about 1.0 pg/ml to about 20 mg/ml.) The upregulation of the biosynthesis of extracellular matrix by dermal fibroblasts cultivated in vitro in a three dimensional collagen foam matrix has been observed and the upregulation of mitotic activity in different cell phenotypes by a variety of tissue-specific extracts has been documented. In addition, wound repair in vitro is highly accelerated in cultures which have been exposed to Signal-plexes, compared with control cultures.

### EXAMPLE 7

Use of Signal-plexes as Pharmaceutical Agents

The extracts alone can be used as pharmaceutical agents. Signal-plexes, acting as pharmaceutical agents, may be deliverable by injection alone or with a carrier (e.g., matrix of hydrated collagen fibers). In a suitable carrier, such as a salve or ointment, the pharmaceutical agent could be applied to an exterior or interior surface of any body part.

### EXAMPLE 8

Use of Signal-plexes in Non-humans

Any of the techniques used in this invention can be applied not only to humans but also to animals of high economic or emotional value, such as race horses and pets respectively.

Equivalents

Those skilled in the art will recognize, or be able to ascertain using no more than routine experimentation, numerous equivalents to the specific procedures described herein. Such equivalents are considered to be within the scope of the present invention and are covered by the following claims. The contents of all references, issued patents, and published patent applications cited throughout this application are hereby incorporated by reference. The appropriate components, processes, and methods of those patents, applications and other documents may be selected for the present invention and embodiments thereof.

What is claimed is:

1. A method for inducing cells to replicate, differentiate, transdifferentiate and or form primoridia in vitro and or tissues or organs in vivo comprising:
a) extracting at least one Signal-plex from a tissue or tissue-specific microparticulates;
b) optionally inducing cells in vitro or in vivo with said Signal-plex to replicate, differentiate, transdifferentiate, and or form specific tissues and or organs; and
c) optionally culturing said cells having said Signal-plex with at least one scaffold in vitro to form primordia in vitro and or said tissues or organs in vivo after implantation of said primordia; and

2. The method of claim 1, wherein said inducing further comprises testing the specificity of said Signal-plex by cells selected from the group consisting of stem cells, cells which may have latent stem cell properties and cells which may be capable of undergoing transdifferentiation.

3. The method of claim 1, further comprising:
a) delivering said Signal-plex to tissues or organs of a recipient after extracting said Signal-plex.

4. The method of claim 3, wherein said delivering is by means of injection.

5. The method of claim 1, further comprising:
a) adding said Signal-plex to a carrier after extracting said Signal-plex; and
b) delivering said carrier to tissues or organs of a recipient.

6. The method of claim 1, further comprising:
a) adding said cells having said Signal-plex attached to said scaffold to a carrier;
b) delivering said carrier to tissues or organs of a recipient. delivering comprises topically applying said carrier onto said tissue or organ of said recipient.

20. The method of claim 5, wherein said carrier is selected from the group consisting of a salve, an ointment and an emollient; and, wherein said delivering comprises topically applying said carrier onto said tissue or organ of said recipient.

21. The method of claim 1, further comprising using said Signal-plex as a pharmaceutical agent by delivering said Signal-plex or said scaffold with or without the use of a carrier to a recipient.

22. The method of claim 21, wherein said delivering is by means of injection.

23. The method of claim 1, further comprising using said Signal-plex to induce wound healing by delivering said Signal-plex or said scaffold with or without the use of a carrier to a wound of a recipient.

24. A pharmaceutical agent produced by the method of claim 1.

25. A topical agent produced by the method of claim 1.

26. A carrier comprising at least one Signal-plex produced by the method of claim 1.

27. A carrier comprising cells having a Signal-plex attached to a scaffold produced by the method of claim 1.

28. Specific cells differentiated or transdifferentiated by the method of claim 1.

29. Specific tissues or organs formed by the method of claim 1.

30. A scaffold comprising specific cells or primordia produced by the method of claim 1.

31. The method of claim 1, wherein said Signal-plex induces histiogenesis and organogenesis in vitro and or in vivo in animals.

32. The method of claim 1, wherein said Signal-plex induces differentiation, transdifferentation, and or said cells to stimulate formation of specific tissues and or organs of the body.

33. A composition comprising scaffold(s), signaling complex(es) and stem cells.
